(19) United States
(12) Patent Application Publication
(10) Pub. No.: US 2026/0168706 A1
(43) Pub. Date: Jun. 18, 2026

(54) COOLING AND HOT COMPRESS DEVICE

(71) Applicant: Reestar International Limited, HONG KONG (HK)

(72) Inventor: Chan Kiu, Hong Kong (HK)

(21) Appl. No.: 19/263,477

(22) Filed: Jul. 8, 2025

(30) Foreign Application Priority Data
Dec. 16, 2024 (CN) 202423100885.0

(51) Int. Cl. F25B 21/04; A61F 7/00

(52) U.S. Cl. CPC F25B 21/04; A61F 7/007; A61F 2007/006; A61F 2007/0075; A61F 2007/0096

(57) ABSTRACT

An example thermal device may include a housing having first and second protruding end portions and a recessed portion between them. The example thermal device may include a heat sink positioned within the recessed portion and occupies at least a majority of the recessed space. Moreover, the heat sink may include a top portion with a recessed region, a second portion thermally coupled to a thermoelectric cooling element, and at least one lateral side directly exposed to ambient air. In some examples, the heat sink may include a recessed region dimensioned to fit one or more fans.

### CROSS-REFERENCE TO RELATED APPLICATIONS

This patent application claims the benefit of and priority to Chinese Utility Patent Application No. 202423100885.0, filed with the Chinese Intellectual Property Office on Dec. 16, 2024, the contents of which are incorporated by reference in its entirety and for all purposes.

### TECHNICAL FIELD

The present invention relates to the technical field of physiotherapy and rehabilitation equipment, and in particular to a thermal device having cryotherapy or thermotherapy functions.

### BACKGROUND

With the improvement of people's living standards, people pay more and more attention to fitness. However, injuries frequently may occur during physical training and cryotherapy and thermotherapy through physiotherapy and rehabilitation equipment may treat or reduce the effects of such injuries. Cryotherapy and thermotherapy are physical therapeutic modalities. As described herein, cryotherapy may reduce localized skin temperature to constrict subcutaneous blood vessels and decrease blood flow. Cryotherapy may alleviate pain and prevent aggravated swelling. Moreover, thermotherapy may promote localized skin blood circulation to dilate blood vessels and accelerate hematoma resolution or relieve muscular pain or tension.

Conventionally, cryotherapy may be typically achieved through cooling using cryotherapy devices, such as ice packs, while thermotherapy may be implemented through heating using thermotherapy devices, such as thermal compresses. In some examples, a thermal device may include thermoelectric elements/devices (e.g., Peltier devices). The thermal device may provide cryotherapy and thermotherapy, allowing for the use of both cryotherapy and thermotherapy through a single device. In such examples, a portable thermal device may include a main housing and a handle housing. The main housing may be configured, in axial sequence from front to rear, with a cooling head, a Thermoelectric Cooler (TEC) element, a heat dissipation module, a ventilation duct, a fan, and a perforated rear cover. A main control board may be disposed on the inner wall of the main housing at the ventilation duct, and the TEC element may be sandwiched between the cooling head and the heat dissipation module. The main control board control the direction of the current supplied to the TEC element to enable cooling or heating functions of the cooling head. A temperature sensor may be further included in the main housing. For example, the temperature sensor may be disposed within an inner groove of the cooling head. A temperature sensor may also be disposed within a groove of the cooling head. Based on the direction of the current supplied by the main control board to the TEC element, the TEC element may switch between cooling and heating modes. Further, rapid heat exchange may be achieved at the periphery of the heat dissipation module through the fan. However, due to the relatively large size and space requirements, such thermal device may fail to meet portability requirements.

In some cases, a thermal device may include features such as, a case, a temperature-controllable element having a first surface and a second surface, a heatsink adjacent to the first surface of the temperature-controllable element, a fan, a thermal diffuser including a first side and a second side that is opposite to the first side and in contact with the second surface of the temperature-controllable element, a support member, and a temperature controller connected to the temperature-controllable element within the case. In such cases, the temperature-controllable element may be a thermoelectric element capable of achieving cooling and heating control. However, the structure of such thermal device may be overly complex and may require the thermal diffuser to contact the temperature-controllable element to accomplish heat transfer, resulting in inconvenient usage. Moreover, the fan, the heatsink, the thermal diffuser, and the temperature-controllable element may be arranged to be in a stacked configuration which may increase the overall size of the thermal device and make it unsuitable for portable use. Further, the fan may be disposed inside the housing, which may result in low heat dissipation efficiency by the fan and affect the heat dissipation effect.

In some instances, a thermal device may require direct skin contact with portions of the user's body. In such instances, the thermal device may need to be tied to the user's body with a strap to prevent them from falling off, so as to ensure normal use. Such a configuration may be inconvenient to use.

### SUMMARY

The disclosed technology provides solutions for addressing the aforementioned issues, including, but not limited to, a thermal device that is compact in size, user-friendly in operation, portable, and facilitates heat dissipation with high heat dissipation efficiency. As described herein, the thermal device may include a housing, a thermoelectric element, a fan, a heatsink, a cover, a main control board, a display, and a battery. In some examples, the housing may be boat-shaped. In some cases, the housing may have protruding portions at two ends of the housing. In some aspects, the housing may include a recessed portion. For example, the recessed portion may be positioned in the middle of the housing. In some instances, the recessed portion of the housing may be between the two ends of the housing. In some examples, the recessed portion may be configured or dimensioned to house the fan and the heatsink. In some cases, the protruding portions (e.g., at the two ends of the housing) may house a control panel, the main control board, and the battery.

As described herein, the fan may be positioned within at the recessed portion. In some examples, the fan may be positioned adjacent and/or thermally coupled to the heatsink. In some instances, the fan may be configured to dissipate or remove heat from the heatsink. Moreover, the heatsink may be positioned within the recessed portion. In some cases, the heatsink may be in contact with or thermally coupled to the fan. Additionally, or alternatively, the thermoelectric element. The heatsink may be configured to perform heat dissipation. For example, the heatsink may be configured to absorb heat generated by the thermoelectric element.

The thermoelectric element (e.g., a Peltier device) may be configured to provide cryotherapy and/or thermotherapy. In some examples, the thermoelectric element may include semiconductor elements of two different types (e.g., n-type and p-type) between two plates—a first plate and a second plate. The plates may be formed out of thermally conductive material (e.g., ceramic plates, such as aluminum oxide ceramic plates, aluminum nitride ceramic plates, beryllium oxide ceramic plates, aluminum plates, copper plates, or any combination thereof). Moreover, the thermoelectric element may provide cryotherapy or thermotherapy based on the direction of the electrical current provided to the thermoelectrical cooling element. For example, when an electric current is applied to the thermoelectrical cooling element, a temperature differential is created across the plates—one plate (e.g., the first plate) absorbs heat and the heat is radiated to and out of the other plate (e.g., the second plate). Reversing the electric current may cause the plate that absorbed heat (e.g., the first plate) to now radiate heat and the plate that radiated heat (e.g., the second plate) to absorb heat. In some cases, a plate of the thermoelectric element may be disposed on or thermally coupled to one side of the heatsink. In some instances, both the thermoelectrical cooling element and the heatsink may be positioned within the recessed portion (e.g., the recessed portion at the middle of the housing).

In some cases, one or more portions of the heatsink may be externally exposed. For example, portion(s) of the heatsink may not be recessed within the housing and/or covered by the housing, such as, the lateral surfaces of the heatsink, including its left and right sides, may be in direct contact with ambient air in a manner similar to the exposed surfaces of the housing itself. This exposed configuration may enable passive and/or active convection to occur without the need for enclosed ventilation paths, ducts, or exhaust vents. Moreover, the exposed configuration of the heatsink may significantly improve heat transfer efficiency by maximizing surface area availability and reducing thermal resistance between the heatsink and the ambient environment.

Additionally, or alternatively, an outer surface of the heatsink may be flush or substantially flush with the outer surface of the housing (e.g. one or more dimensions of the heatsink may be substantially similar or similar to one or more dimensions of the housing). In some instances, a dimensional tolerance of approximately ±1 mm to ±5 mm may exist between the outer surface of the housing and the outer surface of the heatsink, such that the heatsink appears nearly coplanar with the housing in both elevation and lateral profile views. Such configuration may minimize obstruction to ambient airflow and facilitate direct thermal exchange with the surrounding air.

In some aspects, the length of the body of the heatsink may vary depending on design constraints and thermal performance requirements. For example, the heatsink may extend across approximately 40%-60% of the total longitudinal length of the thermal device. In some cases, the length of the heatsink body may be based on available internal space and layout requirements imposed by other electronic components disposed within the housing, such as the display, control button, display screen control board, main control board, thermoelectric element, thermal insulation pad, one or more sensors, battery, and associated wiring paths and mechanical fasteners.

In some instances, the heatsink may include large-sized and exposed heatsink fins. The large-sized and exposed heatsink fins may contribute to more efficient heat dissipation and rapid cooling. As such, such heatsink fins may facilitate heat dissipation with high heat dissipation efficiency.

In some cases, the heatsink may have a concave portion at the middle of the heatsink. In some instances, the fan may be disposed or embedded within the concave portion of the heatsink. Such a configuration may enhance heat dissipation efficiency while reducing occupied space.

In some examples, the cover, such as a metal cover, may be disposed on or thermally coupled to a surface, such as a side surface, of the thermoelectric element. In some instances, the cover may provide structural protection for the thermoelectric element. In some examples, the cover may be formed out of a thermally conductive material (e.g., ceramic cover, such as aluminum oxide ceramic cover, aluminum nitride ceramic cover, beryllium oxide ceramic cover, a metal cover, aluminum cover, copper cover, or any combination thereof). Moreover, the cover may facilitate thermal transfer between the thermoelectric element and an external environment, such as one or more portions of a body of a user, to provide cryotherapy and/or thermotherapy. In some cases, the cover may be disposed on the housing.

In some cases, the main control board may be disposed inside the housing. Moreover, the main control board may be configured to control one or more cooling operations and/or one or more heating operations of the thermoelectric element. As described herein, the cooling operation(s) may provide cryotherapy. Moreover, the heating operation(s) may provide thermotherapy. In some examples, the main control board may control the heating and/or cooling operations of the thermoelectric element based on temperature settings provided through the control panel. In some instances, the thermoelectric element may provide cooling operations and/or heating operations at specific temperatures based on the temperature settings provided through the control panel.

In some aspects, the display may electrically coupled/connected to the main control board. The display may be configured to present temperature information and operational state of the thermoelectrical element (e.g., whether the thermal device is providing cryotherapy and/or thermotherapy). Such displayed information may help prevent adverse events, such as frostbite or burns due to overcooling or overheating. In some examples, the display and the control panel may be combined. For example, the display may include the control panel (e.g., one side of the display). The display and the control panel may be electrically coupled/connected to the main control board to implement cryotherapy and thermotherapy control through the main control board.

In some instances, the battery may be disposed inside the housing. For example, the battery may be positioned at one end of the housing, such as one of the protruding portions of the housing. In some examples, the battery may be electrically coupled or connected to one or more electrical components of the thermal device, such as the display, the main control board, and the thermoelectric element, to power the electrical component(s).

In some examples, the housing and centralized arrangement of the fan and heatsink (e.g., the fan and the heatsink are arranged at the recessed portion at the middle of the housing) may reduce the occupied space of the thermal device and make the cryo-thermal therapy instrument structurally flatter, and more compact size, thereby enhancing portability. Moreover, the thermoelectric element may be thermally coupled to a large-sized heat dissipation apparatus, such as the fan and the heatsink. For example, a top of the thermoelectric element, including a plate that radiates or generates heat, is connected to a large-sized heat dissipation apparatus composed of the fan and the heatsink.

In some cases, the thermal device may include a protective plate. The protective plate may be disposed on the top of the fan, and positioned on the top of the heatsink. In some examples, the heatsink may include structural features, such as a recessed seating region or mechanical alignment surfaces, that may accommodate placement of the protective plate. The structural features may enable the protective plate to be substantially flush with the outer surface of the housing along the vertical (height) direction and/or horizontal (width) direction when positioned and/or engaged with the top of the heatsink. In some instances, the difference in height between the outer surface of the housing and the outer surface of the protective plate when positioned on top of and/or engaged with the heatsink may fall within a margin of ±1 mm to ±5 mm, thereby maintaining a uniform external profile and minimizing protrusions that could interfere with airflow, handling, or device mounting. By maintaining the protective plate in a substantially flush configuration relative to the surrounding housing, the thermal device retains a streamlined geometry suitable for handheld or body-mounted use while optimizing external surface exposure for convective cooling. In some examples, the protective plate and the heatsink may each be formed out of thermally conductive material such as, aluminum alloy, copper, or other metallic materials, to facilitate heat dissipation.

In some aspects, the protruding portions at the two ends of the housing may be a mainboard module chamber and a battery compartment chamber. In some instances, the mainboard module chamber may include a display screen control board and the main control board. As described herein, the display screen control board may be a control board for a display screen. The display screen control board may be electrically coupled or connected to the main control board. In some instances, the battery compartment chamber is provided with a battery pack.

In some instances, the protruding portions at the two ends of the housing may be plastic members. The plastic members may include inwardly protruding snap walls. The inwardly protruding snap walls may be configured to engage with the heatsink, such that the plastic members on left and right sides snap onto the heatsink and secure the heatsink to the housing.

In some examples, the thermal device may include a snap fastener overlaying the housing. The snap fastener may include one or more fastening holes through which a strap may pass through the fastening holes to secure the thermal device to a user's body during use. In some instances, the snap fastener may protrude from the two ends of the housing. In such instances, the snap fastener protruding from the two ends of the housing may include the fastening hole(s). A strap may pass through the fastening hole(s) to secure the thermal device to a human body during use.

In some cases, two ends of the snap fastener may be bent inwardly (e.g., towards each other and/or towards the housing). In such instances, the bent parts of the snap fastener may be protruding from the housing. Moreover, the bent parts may include one or more fastening holes. A strap may pass through the fastening hole(s) to secure the thermal device to a user's body during use.

In some aspects, the snap fastener may include a snap head. The snap head may be disposed on a side surface of the snap fastener. The snap head may extend downward to engage or snap between the heatsink and the housing. The snap head may facilitate secure fixation of the snap fastener to the housing.

In some instances, two or more buttons may be arranged side-by-side on one side of the display screen to enable temperature control. The buttons include dedicated inputs for cryotherapy, thermotherapy, contrast therapy (alternations between cryotherapy and thermotherapy at specific time durations), and switch functions.

In some cases, the thermoelectric element may be surrounded by a thermal insulation pad. In some instances, the thermal insulation pad may be annular. Moreover, the thermoelectric element may be embedded in the thermal insulation pad to prevent heat dissipation from the periphery of the thermoelectric element. Such a configuration may enable unidirectional delivery of cryotherapy and thermotherapy effects.

In some instance, the thermal device may include one or more sensors. In such examples, heating operations and or cooling operations of the thermoelectric element may be based on sensor data generated by the sensor(s). For example, the thermal device may include a temperature sensor, such as a thermometer (e.g., negative temperature coefficient (NTC) thermistor). The temperature sensor may be disposed between the cover contacting the part of the user's body and the thermoelectric element. Sensor data generated by the temperature sensor may indicate temperature information, such as real-time temperatures of the surface of the cover. The main control board (e.g., on or more processors of the main control board) may determine the temperature information based on the sensor data generated by the temperature sensor. The main control board may adjust the current provided to the thermoelectric element to adjust the temperature of the cooling operations and/or the heating operations based on the temperature information. For example, based on preset required temperatures or limited temperatures and the temperature information, the main control board may control the current provided to the thermoelectric element. The temperature of the heating operations and/or cooling operations provided by the thermoelectric element is based on the current provided by the main control board.

### BRIEF DESCRIPTION OF THE DRAWINGS

The accompanying drawings are incorporated herein and form a part of the specification.

FIG. 1 is a perspective view of a thermal device, according to some examples of the present disclosure.

FIG. 2 is a perspective view of the thermal device with a snap fastener, according to some examples of the present disclosure.

FIG. 3 is a front view of the thermal device with a snap fastener, according to some examples of the present disclosure.

FIG. 4 is a side view of the thermal device with a snap fastener, according to some examples of the present disclosure.

FIG. 5 is a rear view of the thermal device with a snap fastener, according to some examples of the present disclosure.

FIG. 6 is an exploded view of the thermal device with a snap fastener, according to some examples of the present disclosure.

FIG. 7 is a schematic diagram of a housing of the thermal device with a snap fastener, according to some examples of the present disclosure.

FIG. 8 is an exploded view of the housing of the thermal device with a snap fastener, according to some examples of the present disclosure.

FIG. 9 is a circuit diagram of a main control board of the thermal device, according to some examples of the present disclosure.

In the drawings, like reference numbers generally indicate identical or similar elements. Additionally, generally, the left-most digit(s) of a reference number identifies the drawing in which the reference number first appears.

### DETAILED DESCRIPTION

Various embodiments and aspects of the present disclosure are described in further detail below with reference to the accompanying drawings. These drawings and the associated descriptions are provided solely for illustrative purposes and are not intended to limit the scope of the disclosure. The specific implementations described herein are provided as examples to facilitate understanding of certain technical features and operational advantages, and it will be understood by those skilled in the art that variations, modifications, and alternatives may be made without departing from the spirit and scope of the disclosure.

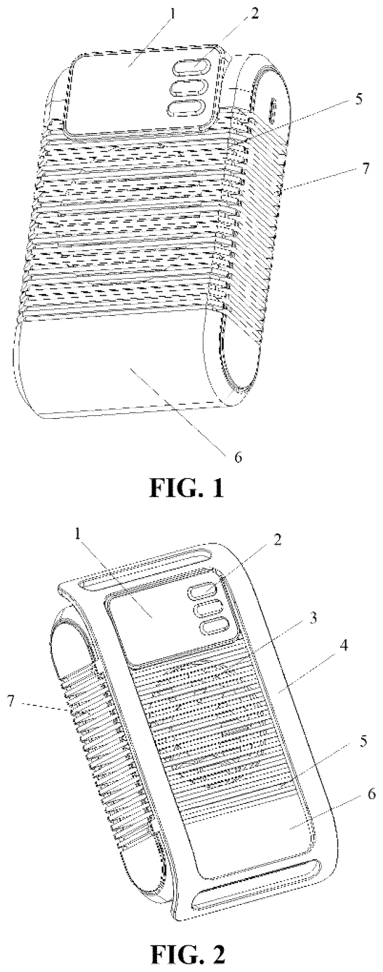

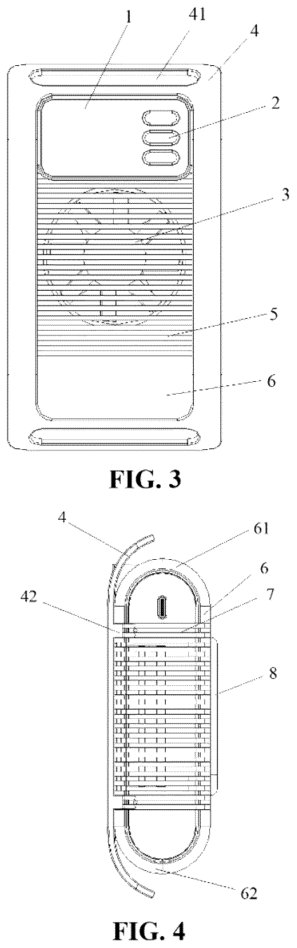

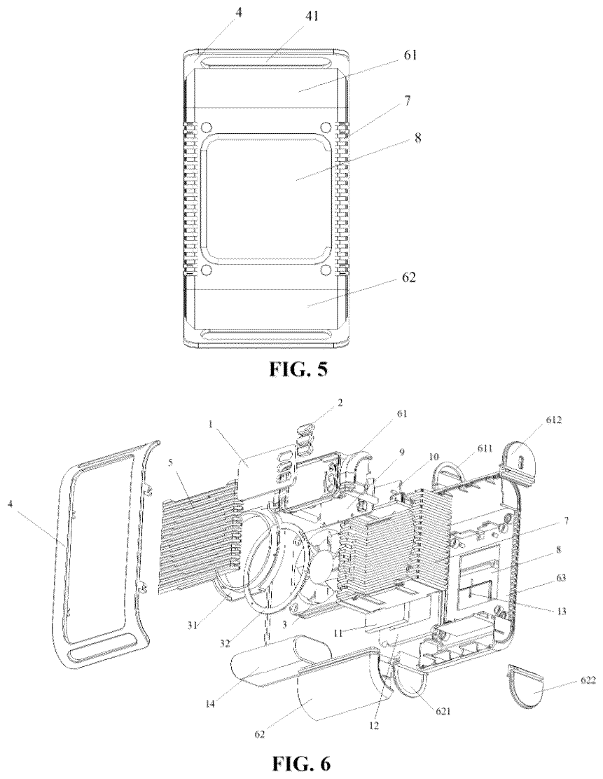

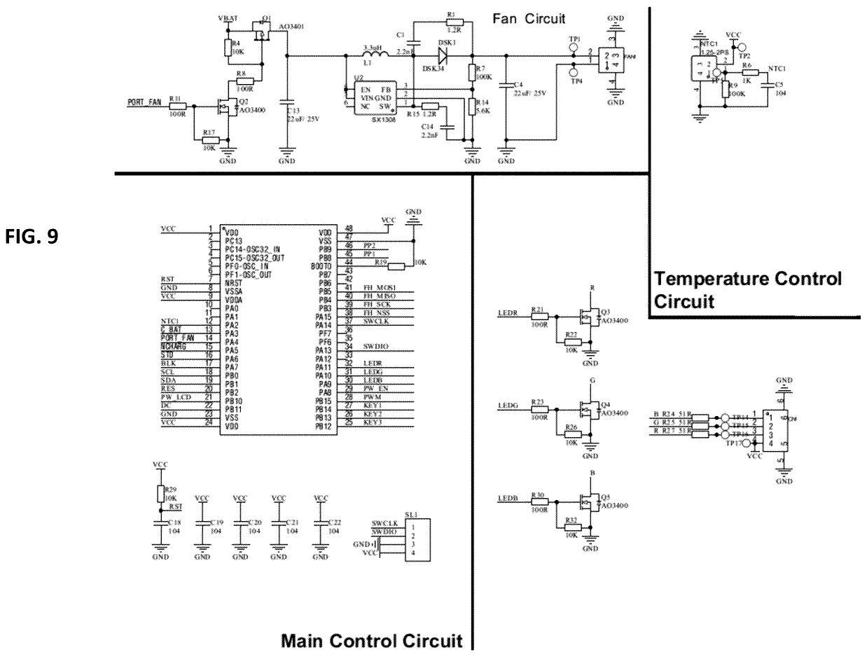

FIGS. 1 to 8 illustrate the thermal device as described herein. The thermal device may include a housing 6, a thermoelectric element 11, a fan 3, a heatsink 7, a cover 8, a main control board 10, a display 1, and a power source 14. As shown in FIG. 1 the snap fastener 4 is not included, while FIG. 2 illustrates the thermal device with the snap fastener 4.

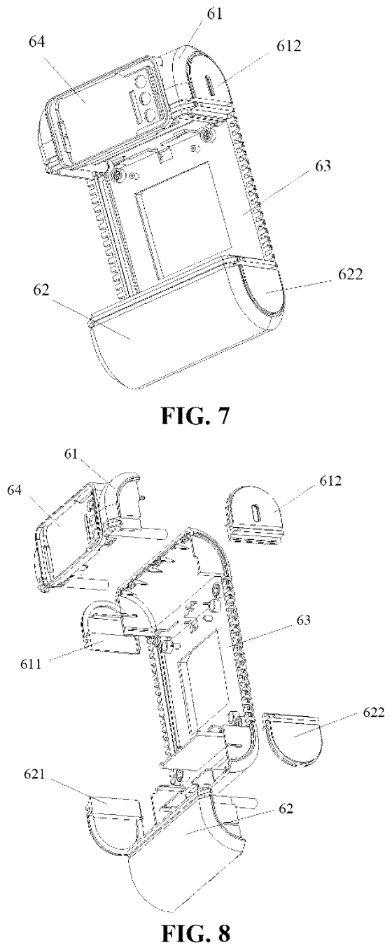

Referring to FIG. 7, the housing 6 may be boat-shaped. In some examples, housing 6 may include one or more protruding portions, such as a protruding portion at each of two ends of housing 6. In some cases, housing 6 may include a recessed portion. In some aspects, the recessed portion may be at the middle portion of the housing 6. For instance, as illustrated in 7, the recessed portion may be between the two ends of housing 6. In some instances, the protruding portion(s) (e.g., at the two ends of the housing 6) may house the display 1, the main control board 10, and the power source 14. In some examples, the recessed portion may be configured or dimensioned to house the fan 3 and the heatsink 7.

As illustrated in FIG. 7, the housing 6 may include an upper end cover 61, a lower end cover 62, a lower cover 63, an upper left side cover 611, an upper right side cover 612, a lower left side cover 621, and a lower right side cover 622. The upper end cover 61, the upper left side cover 611, and the upper right side cover 612 may form a protruding portion, such as a mainboard module chamber. The lower end cover 62, the lower left side cover 621, and the lower right side cover 622 may form another protruding portion, such as a power source compartment chamber. In some examples, the mainboard module chamber may house a display screen control board 9 and/or the main control board 10. As described herein, the display screen control board 9 may be the control board for the display 1. Moreover, the display screen control board 9 may be electrically coupled or connected to the main control board 10. The power source compartment chamber may house the power source 14.

Moreover, as illustrated in FIG. 7, housing 6 may include lower cover 63. Lower cover 63 may be the recessed portion of the housing 6. In this way, space-occupying components are arranged in a separated manner, which helps reduce space usage and facilitates miniaturization of the device. As described herein, the recessed portion of housing 6, such as lower cover 63 may house or be provided with fan 3 and/or the heatsink 7.

In some examples, the protruding portions at the two ends of the housing 6 may include plastic members. The plastic members may include snap walls protruding toward the recessed portion at the middle. In some instances, the plastic members may couple or attach to the heatsink 7 to secure the heatsink 7. For instance, the plastic members of the protruding portions (e.g., plastic members on left and right sides of the housing 6) snap onto the heatsink 7. In some cases, the heatsink 7 may include heatsink fans. Moreover, the edge of the lower cover 63 may include serrations configured to interlock with the heatsink fins of the heatsink 7 to secure the heatsink.

In some cases, the fan 3 may be disposed at the recessed portion of the housing 6. Moreover, the fan 3 may be thermally coupled to the heatsink 7. Further, the fan 3 may be configured to dissipate heat or remove heat absorbed by the heatsink 7. In some instances, the fan 3 may be adjacent to the heatsink 7. In some instances, the heatsink 7 may include a recessed portion dimensioned to fit the fan 3. For instance, the heatsink 7 may include a top portion that includes the recessed portion. The recessed portion may be dimensioned to fit the fan 3. In some aspects, a surface of fan 3 may be flush with or below a surface of a portion of heatsink 7. For instance, a top surface of fan 3 may be flush with or below a surface of a top portion of heatsink 7. In some examples, the fan 3 may be equipped with the light guide cover 31 and/or the light board 32, which are existing structures and thus not elaborated here. Although the figures illustrate a single fan 3, it should be understood that any suitable number of fans may be employed to remove heat from the heatsink 7. For example, the cryo-thermal therapy gun may include two or more fans to enhance airflow and improve heat dissipation efficiency.

In some aspects, a protective plate 5 may be disposed or overlaid on the fan 3. For example, the heatsink 7 may include a top portion. The top portion may include a concaved or recessed portion dimensioned to fit the fan 3. Moreover, the top surface of the fan 3 may be flush with or below the surface of the top portion of heatsink 7, such that the protective plate 5 is also positioned on the top of the heatsink 7 without interfering with the fan 3. Further, the protective plate 5 may be formed out of thermally conductive material such as, aluminum alloy, copper, or other metallic materials, to facilitate heat dissipation or thermally resistive material, such as plastic, to provide structural protection and thermal insulation. As described herein, the protective plate may prevent objects, such as debris, from physical contact with the fan.

As described herein, the heatsink 7 may be disposed at the recessed portion at the middle of the housing 6. Moreover, heatsink 7 may be thermally coupled to and/or be in contact with the fan 3 (e.g., fan 3 may be within a recessed portion of heatsink 7). In some examples, heatsink 7 may be thermally coupled to the thermoelectric element 11. In such examples, heatsink 7 and/or fan 3 may remove heat generated by the thermoelectric element 11. For example, one portion of the heatsink 7, such as a bottom portion (e.g., in some instances the bottom portion may be opposite from the top portion), may be thermally coupled to or in contact with one or more portions of the thermoelectric element 11 that generates heat, while another portion of the heatsink 7, such as a top portion including the recessed portion, may include the fan 3. The heatsink 7 may absorb the heat generated by the portion(s) of the thermoelectric element 11 that generates heat. The fan may remove the heat absorbed by the heatsink 7 and direct it away from heatsink 7. In some examples, the heatsink 7 may be formed out of thermally conductive material such as, aluminum alloy, copper, or other metallic materials, to facilitate heat dissipation.

In some examples, the heatsink 7 may occupy almost the entire recessed portion space of the housing 6 or at least the majority of the recessed portion space of the housing 6. Additionally, or alternatively, one or more portions of heatsink 7 may be externally exposed (e.g., the portion(s) of heatsink 7 may not be recessed within housing 6 or covered by housing 6). For instance, one or more lateral portions of heatsink 7, such as the left side, right side, front side and/or rear side of the heatsink 7, may be in direct contact with ambient air in a manner similar to the exposed surfaces of the housing 6 itself. As described herein, this exposed configuration permits passive and active convection to occur without the need for enclosed ventilation paths, ducts, or exhaust vents.

In some cases, an outer surface of the heatsink 7, such as one or more surfaces of the lateral portion(s) of heatsink 7, such as the left side and/or right side of the heatsink 7, may be substantially flush with the outer surface of the housing 6. In such cases, one or more dimensions of the heatsink 7 may be similar or substantially similar to the dimensions of the housing 6 (e.g. the width and height dimensions of the heatsink 7 may be substantially similar or similar to the width and height dimensions of the housing 6). In some instances, a dimensional tolerance of approximately ±1 mm to ±5 mm may exist between the outer surface of the housing 6 and the outer surface of the heatsink 7, such that the heatsink 7 appears nearly coplanar with the housing 6 in both elevation and lateral profile views. In some examples, a width of a top portion of the heatsink 7 may equal to or greater than the width of a top portion of the housing 6.

In some aspects, the length of the body of the heatsink 7 may vary depending on design constraints and thermal performance requirements. For example, the heatsink 7 may extend across approximately 40% to 60% of the total longitudinal length of the thermal device. In some cases, the length of the boy of the heatsink 7 may be based on available internal space and layout requirements imposed by other electronic components disposed within the housing 6, such as the display 1, control button 2, display screen control board 9, main control board 10, thermoelectric element 11, thermal insulation pad 12, NTC thermistor 13, battery 14, and associated wiring paths and mechanical fasteners.

In some aspects, the heatsink 7 may include structural features, such as a recessed seating region or mechanical alignment surfaces, that may accommodate placement of the protective plate 5. The structural features may enable the protective plate 5 to be substantially flush with the outer surface of the housing 6 along the vertical (height) direction and/or horizontal (width) direction when positioned and/or engaged with the top of the heatsink 7. In some instances, the difference in height between the outer surface of the housing 6 and the outer surface of the protective plate 5 when positioned on top of and/or engaged with the heatsink may fall within a margin of ±1 mm to ±5 mm.

As described herein, the thermoelectric element 11 may be configured to provide cryotherapy and thermotherapy. In some examples, the thermoelectric element 11 may include semiconductor elements of two different types (e.g., n-type and p-type) between two plates—a first plate and a second plate. The plates may be formed out of thermally conductive material (e.g., ceramic plates, such as aluminum oxide ceramic plates, aluminum nitride ceramic plates, beryllium oxide ceramic plates, aluminum plates, copper plates, or any combination thereof). Moreover, the thermoelectric element 11 may provide cryotherapy or thermotherapy based on the direction of the electrical current provided to the thermoelectrical cooling element. For instance, when an electric current is applied to the thermoelectrical cooling element 11, a temperature differential is created across the plates—one plate (e.g., the first plate) absorbs heat and the heat is radiated to and out of the other plate (e.g., the second plate). Reversing the electric current may cause the plate that absorbed heat (e.g., the first plate) to now radiate heat and the plate that radiated heat (e.g., the second plate) to absorb heat. In some cases, a plate of the thermoelectric element 11 may be being disposed on or thermally coupled to one side of the heatsink. In some instances, both the thermoelectrical cooling element 11 and the heatsink 7 may be positioned within the recessed portion (e.g., lower cover 63). In some cases, the thermoelectric element 11 is a TEC cooling element. The TEC cooling element may include an anode that employs a processed aluminum alloy cold-hot plate. The anode may be durable and easy to clean.

In some aspects, the thermoelectric element 11 may be surrounded by a thermal insulation pad 12. As shown in FIG. 5, the thermal insulation pad 12 may be annular. Moreover, the thermoelectric element 11 may be embedded in the thermal insulation pad 12 to prevent heat dissipation from the periphery of the thermoelectric element 11. Such a configuration may enable unidirectional delivery of cryotherapy and thermotherapy effects. In some instances, the thermoelectric element 11 and the thermal insulation pad 11 may be disposed on the inner side surface of the lower cover 63.

In some examples, a cover 8 may be thermally coupled to or overlaid on the thermoelectric element 11, such as a side surface of thermoelectric current 11 (e.g., a plate of the thermoelectric 11 that is not thermally coupled to the heatsink 7). Additionally, and/or alternatively, cover 8 may be overlaid on the thermal insulation pad 12. In some instances, the cover 8 may be formed out of thermally conductive material, such as a ceramic cover (e.g., aluminum oxide ceramic cover, aluminum nitride ceramic cover, and/or a beryllium oxide ceramic cover), a metal cover (such as a, aluminum cover or copper cover), or a thermally conductive and malleable material, such as silicone, or any combination thereof. As described herein, cover 8 may have excellent thermal conductivity and can transfer cryotherapy and thermotherapy effects to the external environment, such as portions of a user's body. In some cases, the cover 8 can protect the thermoelectric element 11. Additionally, or alternatively, the cover 8 may be disposed on the lower cover 63.

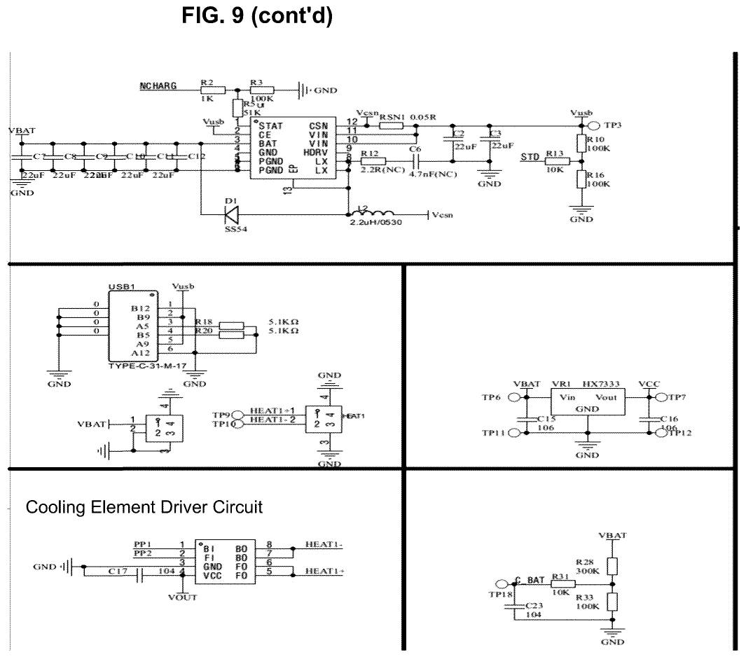

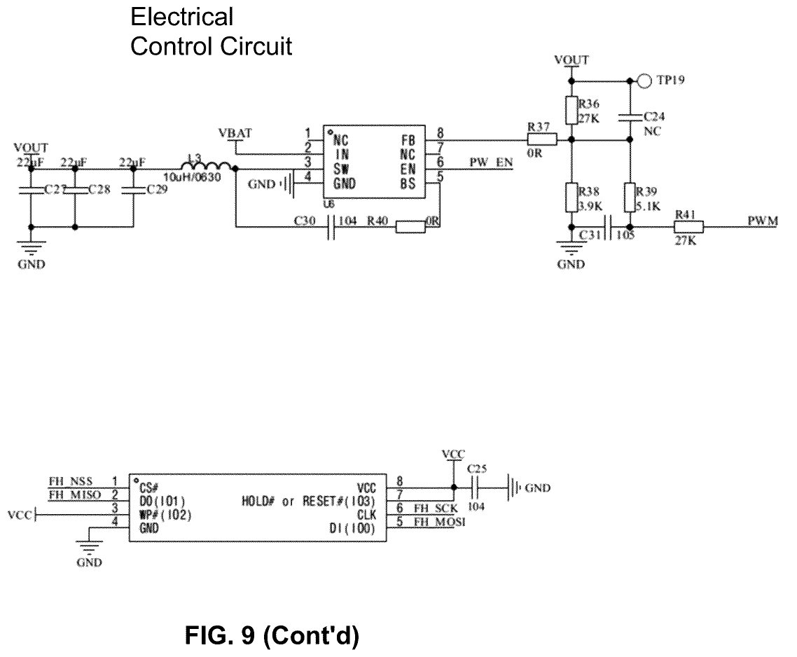

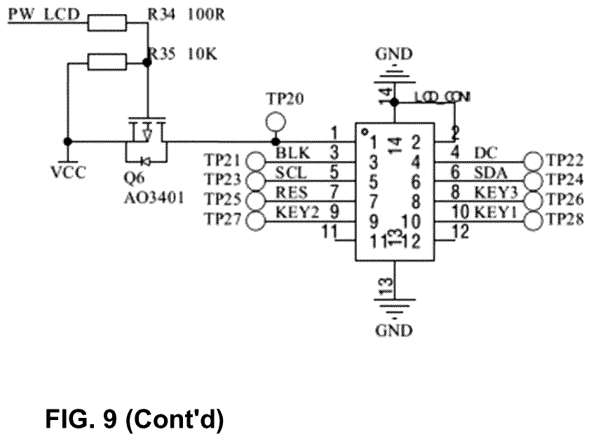

The main control board 10 may be disposed inside the housing 6. Moreover, the main control board 10 may be configured to control the cooling and heating control of the thermoelectric element 11. In some examples, the main control board 10 may control the heating and/or cooling operations of the thermoelectric element based on temperature settings provided through one or more control buttons 2. In some instances, the thermoelectric element may provide cooling operations and/or heating operations at specific temperatures based on the temperature settings provided through the control panel. The control circuit of the main control board is as illustrated in FIG. 9.

The display 1 may electrically coupled to or connected to the main control board 10. The display 1 may be configured to present temperature information and operational state of the thermoelectrical element 11 (e.g., whether the thermal device is providing cryotherapy and/or thermotherapy). In some examples, the display 1 and the control switch 2 may be combined. For example, the display 1 may include control switch 2 (e.g., a control panel for the thermal device). As illustrated in at least FIGS. 1, 2 and/or 6, control switch 2 on one side of the display 1. As described herein, the control switch 2 may be electrically connected to or coupled to the main control board 10 to cause the thermoelectric element 11 implement cryotherapy and/or thermotherapy operations through the main control board 10. For example, the display 1 may be connected to a display screen control board 9. The display screen control board 9 maybe electrically connected to or coupled to the main control board 10, and in some instances, the display screen control board 9 may be integrated with the main control board 10. The display screen control board 9 can cause display 1 to display the temperature information and/or the operational state of the thermoelectrical element 11 based on data received from the main control board 10 (e.g., sensor data from a temperature sensor and/or state data characterizing the operational state of the thermoelectrical element 11).

The power source 14, such as a battery, may be disposed inside the housing 6. For example, the power source 14 may be positioned at one end of the housing, such as batter compartment chamber. Additionally, the power source 14 may be electrically connected to one or more electrical components of the thermal device, such as the display 1, the main control board 10, the thermal electrical cooling element 11 to power the electrical component(s).

In some examples, the thermal device may include snap fastener 4. The snap fastener 4 may be configured to secure the thermal device to a portion of a body of the user. For example, the snap fastener 4 may be overlaid on housing 6. In some instances, the snap fastener 4 may be overlaid on the protective plate 5 and the housing 6 above the fan 3. Moreover, is the snap fastener 4 may be provided with one or more fastening holes 41. An attached strap may pass through the fastening hole(s) 41 to secure the thermal device to a body of the user. In some cases, the snap fastener 4 may protrude from the two ends or protruding parts of the housing 6. Moreover, the snap fasteners 4 protruding from the two ends are provided with the fastening hole(s) 41. The fastening hole(s) 41 may be configured to allow a strap to pass through to secure the thermal device to the user's body during use of the thermal device.

In some aspects, two ends of the snap fastener 4 are inwardly bent (e.g., towards each other and/or towards the housing). In such instances, the bent parts protruding from the housing 6 may include the fastening hole(s) 41. A strap may pass through the fastening hole(s) 41 to secure the thermal device to a user's body during use of the thermal device.

In some instances, snap fastener 4 may include a snap head 42. In some cases, the snap head 42 may be disposed on a side surface of the snap fastener 4. Moreover, the snap head 42 may extend downward to engage with or snap between the heatsink 7 and the housing 6 to facilitate secure fixation of the snap fastener 4.

In some examples, the upper portion of the upper end cover 61 may be provided with a display slot 64. Additionally, the display 1 may be disposed within the display slot 64. Moreover, control buttons 2 may be positioned or arranged on a side surface of display 1. The display screen of display 1 may be configured to display real-time temperatures of cryotherapy and/or thermotherapy provided by the thermoelectric element 11. The displayed real-time temperatures may enable real-time feedback of the current temperature provided by the cryotherapy and/or thermotherapy. In some instances, the real-time temperatures may be based on sensor data generated by one or more sensors included in the thermal device. In some cases, the display screen may display temperature information and/or timing information (e.g., time remaining of a thermotherapy and/or cryotherapy provided by the thermoelectric element 11.

As described herein, the control buttons 2 may be configured to control the temperature and control the current operational state of the thermoelectric element 11, such as cryotherapy, thermotherapy, contrast therapy, and switch functions. In some cases, the control buttons 2 may include three buttons corresponding to three functions: cryotherapy, thermotherapy, and contrast therapy. In some aspects, to initiate cryotherapy, a user may long-press the cryotherapy button to activate the cryotherapy (e.g., at a default temperature setting). In some instances, when cryotherapy is activated, the indicator lights on the light board 32 will turn blue, with the cryotherapy indicator being lit. In some examples, a user may adjust the temperature setting of the cryotherapy by engaging with the cryotherapy button. For example, a user may short press the cryotherapy button to switch to another preset temperature setting. The user may adjust the temperature subsequently by short pressing the cryotherapy button again to return to the previous temperature setting. In some cases, to power on the device, the user may long-press the cryotherapy button.

In some cases, to initiate thermotherapy, a user may long-press the thermotherapy button to activate thermotherapy (e.g., at a default temperature setting). In some aspects, when thermotherapy is activated, the indicator lights on the light board 32 will turn red, with the red therapy indicator also being lit. In some instances, a user may adjust the temperature setting of the thermotherapy by engaging with the thermal therapy button. For example, a user may short press the thermotherapy button to switch to another preset temperature setting. The user may adjust the temperature subsequently by short pressing the thermotherapy button again to return to the previous temperature setting. In some cases, to power on the device, the user may long-press the thermotherapy button.

In some examples, to initiate contrast therapy, which includes alternations between cryotherapy and thermotherapy at specific time durations (e.g., 5 minutes of cryotherapy and 3 minutes of thermotherapy), long-press the contrast therapy button to activate the contrast therapy. In some cases, when contrast therapy is activated, the indicator lights will switch between blue and red to denote which of the different therapies is currently occurring (e.g., cryotherapy or thermotherapy). In some instances, the contrast therapy indicator also being lit, while the indicator lights switch between blue and red. In some aspects, to power on the device, the user may long-press the contrast therapy button.

In some aspects, the thermal device may include one or more sensors 13. In such examples, heating operations for the thermotherapy and or cooling operations for the cryotherapy of the thermoelectric element may be based on sensor data generated by the sensor(s) 13. Additionally, or alternatively, the temperature information displayed by display 1 may be based on the sensor data generated by the sensor(s) 13. For example, the thermal device may include a temperature sensor, such as a thermometer (e.g., a negative temperature coefficient thermistor (NTC)). The sensor(s) 13 may be disposed between the cover 8 contacting a portion of a user's body and the thermoelectric element 11. During the operational state (e.g., cryotherapy and/or thermotherapy), the sensor(s) 13 continuously measures the temperature of the cryotherapy or thermotherapy surface/plate in contact with the body part. The sensor(s) 13 may generate sensor data including temperature information characterizing the measured temperatures. The sensor data generated by the sensor(s) 13 may be transferred to the main control board 10. The main control board 10 (e.g., one or more processors of the main control board) may determine the temperature information based on the sensor data generated by the temperature sensor. The main control board 10 may adjust the current provided to the thermoelectrical cooling element 11 to adjust the temperature of the cooling operations and/or the heating operations based on the temperature information. Additionally, or alternatively, the main control board 10 may adjust the current provided to the thermos electrical cooling element 11 based on preset required temperatures or limited temperatures. For example, the preset temperature is a minimum of 4° C. or a maximum temperature of 53° C. Based on the sensor data, the main control board 10 may determine whether and when these upper or lower limits are reached. Upon such upper or lower limits being reached the main control board 10 may control the current to prevent its temperature from exceeding this operational range.

In some instances, the housing 6 may define an asymmetrically positioned recessed portion. For instance, the recessed portion may be located closer to one end of the housing 6, rather than being centrally positioned along the longitudinal axis of the thermal device. In such instances, the heatsink 7, fan 3, and in some instances the thermoelectric cooling element 11, may also be correspondingly shifted toward the asymmetrically located recessed portion.

For example, the heatsink 7 may be positioned within the asymmetrically position recessed portion and at least a first lateral portion or side of the heatsink 7, such as a left side, and a second lateral side or portion of the heatsink 7, such as a right side, may be exposed (e.g., to ambient air, as described herein). A third lateral side or portion of the heatsink 7, such as a front-facing or rear-facing side, may also be exposed (e.g., to ambient air, as described herein). For example, the heatsink 7 may have left, right, and front sides exposed, or the heatsink 7 may have left, right, and rear sides exposed. These exposed surfaces may allow passive or active convection to occur without relying on internal airflow ducts, exhaust vents, or forced channeling mechanisms. Moreover, the fan 3 may be disposed within a recessed portion of the heatsink 7, such as a top portion of heatsink 7. Further, the thermoelectric cooling element 11 may be thermally coupled to a surface of the heatsink 7 that is not exposed (e.g., a bottom surface or portion of heatsink 7). In such an example, and in some instances, the housing 6 may include one protruding end portion (e.g., a first protruding end portion or a second protruding end portion).

In some cases, the thermal device may further include one or more vibration components (not illustrated in FIGS. 1-9) configured to perform vibrational operations. The vibration component(s) may include, but are not limited to, an electric or electronic motor having an off-centered mass attached to a rotational shaft of the motor, a magnet-based oscillating motor, a magnet-based vibrating diaphragm motor, or any combination thereof. The vibrational operations may be performed independently of the cryotherapy and/or thermotherapy functions of the thermal device or concurrently while cryotherapy and/or thermotherapy is being applied. For example, vibrational operations may be activated prior to, during, or after thermal treatment to stimulate blood flow, relax muscle tissue, or enhance thermal transfer efficiency.

In some instances, the vibrational operations may include one or more predetermined or user-selectable vibration patterns, frequencies, and/or durations. For instance, the thermal device may perform pulsed or continuous vibrations at varying intensities or frequencies depending on user settings or therapeutic objectives. In some aspects, the vibration component(s) may be controlled by the main control board 10, which may regulate activation timing, repetition, and modulation of the vibrational signals based on stored profiles or manual input received via the control panel or associated user interface.

In some examples, a plurality of thermal devices or multiple thermal devices may be operatively coupled to deliver localized or regional cryotherapy and/or thermotherapy to a larger area or multi-surface portion of a user's body. For example, multiple thermal devices may each be coupled to and spatially arranged on a shared substrate or support structure, such as a flexible wrap, band, brace, or garment. The multiple thermal devices on a shared support structure may conform to the contours of anatomical regions such as the user's knee, shoulder, thigh, or back.

In some aspects, the shared substrate or support structure may be from flexible, durable, and thermally insulative or conductive materials (e.g., neoprene, polyurethane, silicone, nylon-elastane blends, or multilayer composite textiles). In some cases, the support structure may include device-specific cavities, pockets, or mounting frames that retain each thermal device in a fixed position relative to a target region of the user's body, while still permitting localized adjustment or repositioning. In some instances, the support structure may further include elastic straps, hook-and-loop fasteners, snap mechanisms, and/or integrated tensioning systems to secure the entire assembly (e.g., the multiple thermal devices on the shared substrate or support structure) to the user's body during use. In some examples, the support structure may be contoured or pre-curved to anatomically wrap around curved body regions (e.g., the lateral and anterior aspects of the knee or the deltoid and upper trapezius regions of the shoulder) to maintain consistent contact between each thermal device and the user's skin or clothing layer.

For example, the multiple thermal devices on a shared substrate or support structure may be configured for a knee. In such a configuration, three or more thermal device may be arranged triangularly or circumferentially around the knee joint. In another example, the multiple s on a shared substrate or support structure may be configured for a shoulder. In such a configuration, a distributed array of thermal devices may be positioned such that when placed on the user, the distributed array of thermal devices may be positioned across the deltoid, scapular, and clavicular regions to deliver targeted thermal therapy while conforming to the complex topography of the shoulder.

In some cases, a control interface may be operatively or communicatively coupled to the each of the multiple thermal devices of the assembly to enable synchronous or independent control of each of the multiple thermal devices. In some instances, the control interface may include a display, a central control panel and/or one or more input buttons. Moreover, the control interface may allow the user to selectively activate cryotherapy, thermotherapy, or vibrational functions for individual thermal devices or grouped subsets thereof. Further, the control interface may enable users to customize parameters such as temperature setpoint, therapy duration, vibrational pattern, and vibrational intensity and/or frequency across the multiple thermal devices. In some examples, the control interface may be physically located on one of the thermal devices or on another component or device attached or coupled to the shared substrate or support structure. Additionally, or alternatively, the control interface may be remotely located (e.g., integrated into a wireless handheld controller or mobile device application) and in communication with the multiple thermal devices via wired or wireless communication protocols (e.g., BLE, Wi-Fi).

In some aspects, the display of the control interface may present temperature-related information and operational status for one or more of the multiple thermal devices. For example, the temperature information may be based on sensor data obtained from one or more temperature sensors associated with one or more of the thermal devices. The operational status may include an indication of whether a particular thermal device is actively performing cryotherapy, thermotherapy, or is in an idle or standby mode. Such display of real-time status and temperature data may enable users to monitor and verify the ongoing operation of each device and avoid potential issues such as overcooling, overheating, or inadvertent non-activation of a device.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments but should be defined only in accordance with the following claims and their equivalents.

Claim language or other language in the disclosure reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

Illustrative examples of the disclosure include:
Aspect 1. A thermal device, comprising a housing, a thermoelectric cooling element, a fan, a heatsink, a metal cover, a main control board, a display, and a battery, wherein the housing is boat-shaped with protruding portions at two ends and a recessed portion at the middle, the two ends of the boat-shaped housing being respectively provided with a control panel, the main control board, and the battery, and the recessed portion at the middle being provided with the fan and the heatsink; the fan is disposed at the recessed portion at the middle of the housing and adjacent to the heatsink, and configured to dissipate heat from the heatsink; the heatsink is also disposed at the recessed portion at the middle of the housing and is in contact with the fan and the thermoelectric cooling element, and configured to perform heat dissipation; the thermoelectric cooling element is capable of cooling while heating, the thermoelectric cooling element being disposed on one side of the heatsink and positioned at the recessed portion at the middle of the housing; the metal cover is disposed on a side surface of the thermoelectric cooling element and the metal cover is disposed on the housing, and the metal cover has high thermal conductivity to rapidly transfer cryotherapy and thermotherapy effects to the external environment; the main control board is disposed inside the housing, and is configured to implement cooling and heating control of the thermoelectric cooling element and implement specific temperature control; the display is configured to implement display of temperature control, and has a control switch on one side, the display and the control switch being electrically connected to the main control board to implement cryotherapy and thermotherapy control through the main control board; and the battery is disposed inside the housing and is positioned at one end of the boat-shaped housing, and is electrically connected to the display and the main control board to power the display and the main control board.
Aspect 2. The thermal device of Aspect 1, wherein the heatsink has a concave portion at the middle, in which the fan is disposed.
Aspect 3. The thermal device of Aspect 2, wherein a protective plate is disposed on the top of the fan, and the protective plate is positioned on the top of the heatsink, the protective plate and the heatsink being both made of metal material.
Aspect 4. The thermal device of any of Aspects 1 to 3, wherein the protruding portions at the two ends of the housing are a mainboard module chamber and a battery compartment chamber, wherein the mainboard module chamber has a display screen control board and the main control board, the display screen control board being a control board for a display screen and electrically connected to the main control board, and the battery compartment chamber is provided with a battery pack.
Aspect 5. The thermal device of Aspect 4, wherein the protruding portions at the two ends of the housing are plastic members, the plastic members having inwardly protruding snap walls, such that the plastic members on left and right sides snap onto the heatsink.
Aspect 6. The thermal device of any of Aspects 1 to 5, further comprising a snap fastener overlaying the housing, wherein the snap fastener is provided with fastening holes, an attached strap passing through the fastening holes for securement.
Aspect 7. The thermal device of Aspect 6, wherein the snap fastener protrudes from the two ends of the housing, the protruding parts being provided with the fastening holes.
Aspect 8. The thermal device of Aspect 7, wherein two ends of the snap fastener are inwardly bent, the bent parts protruding from the housing and being provided with the fastening holes; and a snap head is further disposed on a side surface of the snap fastener, the snap head extending downward and snapping between the heatsink and the housing.
Aspect 9. The thermal device of any of Aspects 1 to 8, wherein the thermoelectric cooling element is peripherally surrounded by a thermal insulation pad, wherein the thermal insulation pad is annular, and the thermoelectric cooling element is embedded in the thermal insulation pad.
Aspect 10. The thermal device of any of Aspects 1 to 9, wherein the device is further provided with a thermometer, which is implemented by an NTC thermistor, the NTC thermistor being disposed between the metal cover and the thermoelectric cooling element.

What is claimed is:

1. A thermal device, comprising:
a housing having a first protruding end portion, a second protruding end portion, and a recessed portion between the first protruding end portion and the second protruding end portion;
a heatsink positioned with in the recessed portion of the housing and occupies at least a majority of the recessed portion of the housing, the heatsink including:
a top portion including a recessed portion,
a second portion, and
at least a first lateral side, wherein one or more portions of the first lateral side is in direct contact with ambient air,
a fan positioned within the recessed portion of the heatsink; and
a thermoelectric cooling element positioned within the recessed portion of the housing and thermally coupled to the second portion of the heatsink.

2. The thermal device of claim 1, wherein a width of the top portion is equal to or greater than the width of a top portion of the housing.

3. The thermal device of claim 1, wherein at least the first lateral side is substantially flush with a corresponding outer surface of the housing.

4. The thermal device of claim 3, wherein a surface of at least the first lateral side is substantially flush with a corresponding outer surface of the housing within a dimensional tolerance of approximately ±1 mm to ±5 mm.

5. The thermal device of claim 1, wherein the heatsink further includes:
a second lateral side, wherein one or more portions of the second lateral side is in direct contact with the ambient air.

6. The thermal device of claim 5, wherein a surface of the second lateral side is substantially flush with a corresponding outer surface of the housing within a dimensional tolerance of approximately ±1 mm to ±5 mm.

7. The thermal device of claim 1, wherein a length of the heatsink extends 40% to 60% of a total longitudinal length of the thermal device.

8. The thermal device of claim 1, wherein the top portion is opposite from the second portion.

9. The thermal device of claim 1, wherein the top portion is adjacent from the second portion.

10. The thermal device of claim 1, further comprising one or more temperature sensors, the one or more temperature sensors being disposed between a cover and the thermoelectric cooling element.

11. The thermal device of claim 1, further comprising:
a cover thermally coupled to the thermoelectric cooling element.

12. The thermal device of claim 1, further comprising:
a protective plate coupled to a surface of the top portion of the heatsink, wherein a surface of the protective plate is substantially flush with a corresponding outer surface of the housing.

13. The thermal device of claim 12, wherein the surface of the protective plate is substantially flush with the corresponding outer surface of the housing within a dimensional tolerance of approximately ±1 mm to ±5 mm.

14. The thermal device of claim 1, further comprising:
a thermal insulation pad covering one or more portions of the thermoelectric cooling element to cause thermal transfer to a unidirectional path.

15. The thermal device of claim 1, wherein:
the heatsink is configured to dissipate heat generated by the thermoelectric cooling element while the thermoelectric cooling element provides cryotherapy without enclosed ventilation ducts, exhaust vents or airflow channels;
the fan is configured to direct airflow across exposed surfaces of the heatsink to enable active heat dissipation; and
the thermoelectric cooling element is configured to provide at least one of cryotherapy and thermotherapy to one or more portions of a body of a user.

16. The thermal device of claim 1, wherein:
the first protruding end portion and the second protruding end portion of the housing are formed from plastic members; and
a plastic member of the first protruding end portion and a plastic member of the second protruding end portion each include an inwardly protruding snap wall configured to engage with the heatsink, the plastic member of the first protruding end portion and the plastic member of the second protruding end portion each secure the heatsink within the recessed portion of the housing.

17. The thermal device of claim 1, further comprising a snap fastener overlaying at least a portion of the housing, the snap fastener includes one or more fastening holes dimensioned to fit a strap and the strap secures the thermal device to a body of a user via the one or more fastening holes.

18. The thermal device of claim 17, wherein:
ends of the snap fastener extend beyond the first protruding end portion of the housing and second protruding end portion of the housing;
the ends are inwardly bent and include the one or more fastening holes; and
the snap fastener further includes a snap head extending downward from a side surface of the snap fastener and configured to engage between the heatsink and the housing.

19. The thermal device of claim 1, further comprising:
a main control board configured to control one or more operations of at least the fan and the thermoelectric element;
a display configured to present at least one of temperature information associated with the thermoelectric element and an operational state of the thermoelectric element, or any combination thereof; and
a power source electrically coupled to the main control board, the fan and the thermoelectric element.

20. The thermal device of claim 19, wherein the first protruding end portion includes a first chamber, and the second protruding end portion includes second chamber, the first chamber houses the main control board and the second chamber houses the power source.
